(19) United States
(12) Patent Application Publication
(10) Pub. No.: US 2018/0169713 A1
(43) Pub. Date: Jun. 21, 2018

(54) METHOD FOR CLEANING A MEMBRANE OF A MEMBRANE AEROSOL GENERATOR, WHICH MEMBRANE CAN VIBRATE, AND COMBINATION OF A CLEANING DEVICE AND A CLEANING LIQUID FOR SUCH CLEANING

(71) Applicant: PARI PHARMA GmbH, Starnberg (DE)

(72) Inventor: Dominik Müller, Kochel (DE)

(73) Assignee: PARI PHARMA GmbH, Starnberg (DE)

(21) Appl. No.: 15/545,591

(22) PCT Filed: Jan. 18, 2016
(86) PCT No.: PCT/EP2016/050881

§ 371 Date: Jul. 21, 2017

(30) Foreign Application Priority Data
Jan. 23, 2015 (DE) 10 2015 201 134.8

(51) Int. Cl. B08B 3/08; A61M 15/00; B08B 7/02; B08B 11/00; B05B 15/55; C11D 3/20; C11D 3/386

(52) U.S. Cl. CPC B08B 3/08; A61M 15/0085; B08B 7/02; B08B 11/00; B05B 17/06; C11D 3/2086; C11D 3/386; A61M 2209/10; B05B 15/55

(57) ABSTRACT

The invention relates to a method for cleaning a membrane (3) of a membrane aerosol generator (4) of an inhalation therapy device (100), which membrane can vibrate, wherein a cleaning liquid is fed to the membrane (3) and the membrane (3) is caused to vibrate in such a way that the cleaning liquid is conveyed through the openings of the membrane (3), wherein the cleaning liquid contains at least one surfactant. The invention further relates to a combination of a cleaning device (1) and of a cleaning liquid, wherein the cleaning device (1) is designed for cleaning a membrane (3) of a membrane aerosol generator (4) of an inhalation therapy device (100), which membrane can vibrate. The cleaning device (1) comprises a liquid-feeding apparatus (2), which is designed to feed the cleaning liquid to the membrane (3), and a vibration activation apparatus (9), which is designed to activate membrane vibrations such that the cleaning liquid is conveyed through the openings of the membrane (3). The cleaning liquid contains at least one surfactant.

The present invention relates to a method for cleaning an oscillatable membrane of a membrane aerosol generator of an inhalation therapy device. The present invention furthermore includes a combination of a cleaning device and a cleaning liquid, whereby the cleaning device is configured for cleaning an oscillatable membrane of a membrane aerosol generator of an inhalation therapy device.

Using inhalation therapy devices, therapeutically effective or medicament-containing liquids are nebulised into an aerosol consisting of respirable particles by means of an aerosol generator. The generated aerosol is offered to a patient for inhalation in the course of an inhalation therapy, as a result of which the therapeutically effective liquid or medicament reaches the respiratory tract of the patient.

In particularly effective inhalation therapy devices, a membrane aerosol generator is used as the aerosol generator for generating the aerosol. The liquid to be nebulised is stored in a storage container and is supplied to the membrane of the membrane aerosol generator on the one side thereof, i.e. the liquid side. If the membrane of the membrane aerosol generator is caused to oscillate, the liquid is transported through openings of the membrane and is released on the other side of the membrane, i.e. the aerosol side, in the form of an aerosol. To cause the membrane to oscillate, the membrane aerosol generator comprises, in addition to the membrane, an oscillation generator, for example an annular piezoelectric element and an annular substrate that are connected to one another and to the membrane in such a manner that an actuation signal supplied to the piezoelectric element triggers oscillation of the oscillation generator, which causes the membrane to oscillate.

As an example of such an inhalation therapy device, EP 1 304 131 A1 discloses an inhalation therapy device with an oscillatable membrane for nebulising a liquid, comprising an oscillation generating device having at least one connecting means for supplying an actuation signal, by means of which the membrane is caused to oscillate such that a liquid disposed on one side of the membrane is nebulised through the membrane and is present on the other side of the membrane as an aerosol.

Inhalation therapy devices with membrane aerosol generators are characterised by a high efficiency, a high dosage accuracy and short therapy times, and thus inhalation therapy devices with membrane aerosol generators are particularly suitable for the application of very expensive and/or highly effective medicaments. In addition, inhalation therapy devices with membrane aerosol generators can be used over a long period of time with satisfactory results. After a large number of therapy sessions, aerosol generation may become slower for a number of reasons (depending on the care with which the required cleaning is carried out, environmental influences, type of medicaments used, etc.), however, this generally does not lead to a deterioration of the aerosol quality, such a droplet size distribution (MMD, GSD).

In almost all cases of use, an excessive extension of the therapy sessions is not acceptable since this could lead to poorer adherence to the prescribed therapy, and the quickest possible administration of a specific dose of the medicament is always desired. The reason for this is that a patient/user of the inhalation therapy device should not be expected to put up with unnecessarily long therapy times. Accordingly, long therapy times are fundamentally undesirable. Furthermore, the comparatively high manufacturing costs of high-quality inhalation therapy devices with membrane aerosol generators must be taken into consideration. There is thus a need to further extend the lifespan of the inhalation therapy device, i.e. the period of time over which an inhalation therapy device with a membrane aerosol generator can be used, without a significant extension of the therapy times and of other properties that are relevant for therapy occurring.

Examinations performed with a scanning electron microscope of membranes used in membrane aerosol generators have shown that in some cases, despite proper cleaning of the membrane aerosol generator, an increasingly large number of the membrane openings sometimes becomes blocked by material of different origins, as a result of which the yield rate (output) of aerosol generation is reduced or the rate of aerosol generation is reduced, which has a negative effect on the duration of the therapy session and disadvantageously extends this.

EP 1 875 936 A1 discloses a method and device for cleaning an oscillatable membrane of a membrane aerosol generator of an inhalation therapy device, whereby the cleaning of the membrane is achieved by a backwashing mode of the membrane. The cleaning liquid is supplied to the membrane on the aerosol release side (in the normal mode) of the membrane and membrane oscillations are subsequently activated, which transport the cleaning liquid through the membrane such that the cleaning liquid drips off on the liquid supply side (in the normal mode) of the membrane.

In view of the above, the object forming the basis for the present invention is to increase the efficiency of cleaning an oscillatable membrane of a membrane aerosol generator of an inhalation therapy device such that the period of time over which the inhalation therapy device can be used with a high standard of quality is extended.

This object is solved by means of a method having the features of claim 1 and by a combination of a cleaning device and a cleaning liquid having the features of claim 9. Advantageous embodiments can be found in the remaining claims.

According to a first aspect, the present invention provides a method for cleaning an oscillatable membrane of a membrane aerosol generator of an inhalation therapy device, wherein a cleaning liquid is supplied to the membrane and the membrane is caused to oscillate in such a manner that the cleaning liquid is conveyed through the openings of the membrane. The cleaning liquid contains at least one surfactant.

The oscillatable membrane can be made of a metal, such as steel, in particular stainless steel, aluminum or another flexible metal, a plastic or a ceramic.

Owing to the use of a cleaning liquid containing at least one surfactant, the efficiency of cleaning the oscillatable membrane can be significantly increased.

The cleaning liquid can contain precisely one surfactant or a plurality of different surfactants.

According to the invention, the cleaning liquid for use in the method according to the invention contains at least one surfactant. In order to achieve an optimal cleaning effect, the concentration of the surfactant or surfactants in the cleaning liquid is preferably 0.05 to 10% by weight, more preferably 0.1 to 5% by weight and particularly preferred 0.5 to 2% by weight, based on the total weight of the cleaning liquid.

The cleaning liquid preferably contains at least one cationic surfactant. The use of cationic surfactants in the method according to the invention has proven to be particularly advantageous since common contaminations on the membrane, such as protein-containing residues, poorly soluble medicament residues as well as dust particles, can thereby be removed from the openings of the membrane in a particularly efficient manner. It was also surprisingly found that the hydrophilic and aerosol-generating properties of the membrane and in particular its permeability for pharmaceutical preparations or fluids remain largely unaffected following the cleaning method according to the invention. This is surprising since cationic surfactants, as is well known, negatively affect the permeability of many membrane materials and are unsuitable in particular for cleaning membrane textiles.

Suitable cationic surfactants are typically quaternary ammonium salts, which may be, for example, chlorides bromides or alkyl sulphates. The cationic surfactants in the cleaning solution may be, for example, surfactants such as di-(C₈₋₂₄)-alkyl dimethyl ammonium chloride or bromide, preferably di-(C₁₂₋₁₈)-alkyl dimethyl ammonium chloride or bromide, for example distearyl dimethyl ammonium chloride or bromide, ditallow alkyl dimethyl ammonium chloride or bromide, dioleyl dimethyl ammonium chloride or bromide, dicoco alkyl dimethyl ammonium chloride or bromide; (C₈₋₂₄)-alkyl dimethyl ethyl ammonium chloride or bromide. Further preferred are (C₈₋₂₄)-alkyl trimethyl ammonium chloride or bromide, preferably cetyl trimethyl ammonium chloride or bromide and (C₂₀₋₂₂)-alkyl trimethyl ammonium chloride or bromide, (C₈₋₂₄)-alkyl dimethyl benzyl ammonium chloride or bromide, preferably (C₁₂₋₁₈)-alkyl dimethyl benzyl ammonium chloride, N-(C₁₂₋₁₈)-alkyl pyridinium chloride or bromide, preferably N-(C₁₂₋₁₆)-alkyl pyridinium chloride or bromide, N-(C₁₀₋₁₈)-alkyl isoquinolinium chloride, bromide or monoalkyl sulphate, N-(C₁₂₋₁₈)-alkyl polyoylaminoformylmethyl pyridinium chloride, N-(C₁₂₋₁₈)-alkyl-N-methyl morpholinium chloride, bromide or monoalkyl sulphate, N-(C₁₂₋₁₈)-alkyl-N-ethyl morpholinium chloride, bromide or monoalkyl sulphate; (C₁₆₋₁₈)-alkyl pentaoxethyl ammonium chloride or diisobutyl phenoxy ethoxy ethyl dimethyl benzyl ammonium chloride. Particularly preferred are, inter alia, benzalkonium chloride, didecyl dimethyl ammonium chloride, dioctyl dimethyl ammonium chloride, cetrimonium bromide, cetylpyridinium chloride (hexadecylpyridinium chloride), dimethyl decyloxethyl ammonium propionate, N,N-didecyl-N-methyl-poly(oxyethyl) ammonium propionate and isotridecanol ethoxylate.

The cationic surfactant in the cleaning solution preferably also comprises bactericidal and/or anti-fungal and/or anti-viral properties such that the membrane of the membrane aerosol generator can also be disinfected to the greatest possible extent by the method according to the invention. This is advantageous in particular if the inhalation therapy device is used for the treatment of infectious diseases of the lungs.

The concentration of the cationic surfactant or cationic surfactants in the cleaning liquid is preferably in the range of 0.03 to 10% by weight, more preferred 0.07 to 4% by weight, particularly preferred 0.3 to 2% by weight, based on the total weight of the cleaning liquid.

In a further embodiment, the cleaning liquid contains at least one nonionic surfactant. The nonionic surfactants used are preferably alkoxylated, advantageously ethoxylated, in particular primary alcohols having preferably 8 to 18 C atoms and, on average, 1 to 12 mol of ethylene oxide (EO) or propylene oxide (PO) per mol of alcohol, in which the alcohol radical can be linear or preferably methyl-branched in the 2-position or can contain a mixture of linear and methyl-branched radicals, such as are typically present in oxo alcohol radicals. However, in particular alcohol ethoxylates with linear radicals of alcohols of a native origin having 12 to 18 C atoms, for example from coco, palm, tallow fatty or oleyl alcohol, and an average of 2 to 8 EO per mol of alcohol are preferred. The preferred ethoxylated alcohols include, for example, C₁₂₋₁₄ alcohols with 3 EO, 4 EO or 7 EO, C₉₋₁₁ alcohol with 7 EO, C₁₃₋₁₅ alcohols with 3 EO, 5 EO, 7 EO or 8 EO, C₁₂₋₁₆ alcohols with 5 EO, C₁₂₋₁₈ alcohols with 3 EO, 5 EO or 7 EO and mixtures thereof, such as mixtures of C₁₂₋₁₄ alcohol with 3 EO and C₁₂₋₁₈ alcohol with 7 EO. The indicated degrees of ethoxylation represent statistical averages that can be an integer or a fractional number for a specific product. Preferred alcohol ethoxylates have a narrowed homologue distribution. In addition to these nonionic surfactants, fatty alcohols with more than 12 EO can also be used. Examples hereof are tallow fatty alcohol with 14 EO, 25 EO, 30 EO or 40 EO. Nonionic surfactants that comprise EO and PO groups together in the molecule can also be used according to the invention. Block copolymers with EO-PO block units or PO-EO block units can hereby be used, as can EO-PO-EO copolymers or PO-EO-PO copolymers. Mixed alkoxylated nonionic surfactants, in which EO and PO units are distributed statistically rather than blockwise, can obviously also be used. Such products can be obtained by the simultaneous action of ethylene oxide and propylene oxide on fatty alcohols.

In particular EO derivatives such as PEG-90, PEG-150, PEG-180 or ceteareth, such as ceteareth-80, must be cited as examples of suitable nonionic surfactants.

The concentration of the nonionic surfactant or nonionic surfactants in the cleaning liquid is preferably in the range of 0.01 to 5% by weight, more preferred 0.03 to 3% by weight, particularly preferred 0.1 to 1% by weight, based on the total weight of the cleaning liquid.

In one embodiment, the cleaning liquid comprises at least one cationic surfactant in combination with at least one nonionic surfactant. The use of such a combination of surfactants in the cleaning liquid allows a particularly efficient simultaneous removal of residues on the membrane having different physical properties, and thus a particularly efficient cleaning of the membrane.

Instead of or in addition to the cationic and/or nonionic surfactants, the cleaning liquid can also contain at least one anionic surfactant. Used as anionic surfactants are, for example, those of the sulphonate and sulphate types. C₉₋₁₃ alkyl benzene sulphonates, olefin sulphonates, i.e. mixtures of alkene and hydroxyalkane sulphonates, as well as disulphonates, come into question as surfactants of the sulphonate type. Also suitable are alkane sulphonates. The esters of α-sulfo fatty acids (ester sulphonates), for example the α-sulphonated methyl esters of hydrogenated coconut, palm kernel or tallow fatty acids, are likewise also suitable.

The cleaning liquid can furthermore contain at least one acid. The cleaning liquid can contain precisely one acid or a plurality of different acids.

By using a cleaning liquid containing at least one acid, in particular contaminations with lime and blockages in the membrane caused by lime can be removed in a particularly effective manner. Furthermore, the surface of the membrane can be passivated by the at least one acid contained in the cleaning liquid so as to thus reduce the likelihood of future contaminations and blockages of the membrane.

The at least one acid can be an organic acid, in particular citric acid or acetic acid.

The cleaning liquid for the method according to the invention preferably has a pH value of between 4 and 8, more preferred of between 5 and 7.5 and particularly preferred of between 5.5 and 7. The desired pH value of the cleaning liquid can be achieved, for example, in that the cleaning liquid contains at least one acid and/or at least one buffer.

Non-toxic, water-soluble, organic or inorganic acids can be used as acids for the cleaning liquid, with which the desired pH value of the cleaning liquid can be set. The use of organic acids is particularly preferred since owing to the buffer effect thereof, possibly in the presence of the salts of these acids, the pH value of the cleaning liquid can be kept in the desired range during cleaning.

This is advantageous in particular if the membrane has been contaminated by basic or acidic residues.

In particular organic acids, such as formic acid, acetic acid, citric acid, tartaric acid, malic acid or sulfamic acid must be cited as preferred acids for use in the cleaning liquid. The use of citric acid in the cleaning liquid is particularly advantageous since an excellent cleaning effect is hereby achieved. Citric acid as well as the salts thereof furthermore has a very good buffer effect at preferred pH values.

In order to increase the buffer effect of the cleaning liquid, additional buffers such as sodium citrate or sodium acetate can be added hereto.

The cleaning liquid can in particular contain one or more buffers to adjust the pH value of the cleaning liquid. The use of one or more such buffers is also advantageous in particular in cleaning liquids that furthermore contain at least one enzyme. By means of the buffer or buffers, the pH value of the cleaning liquid can be adjusted to the range in which the enzyme exhibits its maximum effect, for example for cleaving organic compounds, such as proteins and/or peptides. Contaminations and blockages of the membrane can thus be removed in a particularly effective manner.

The cleaning liquid can furthermore contain at least one enzyme. The cleaning liquid can contain precisely one enzyme or a plurality of different enzymes.

Owing to the use of a cleaning liquid containing at least one enzyme, in particular organic contaminations and blockages of the membrane can be removed particularly effectively. The efficiency of the cleaning of the membrane can be further increased in this manner.

The at least one enzyme can be an enzyme for cleaving proteins and/or peptides.

Particularly preferred enzymes for use in the cleaning liquid are in particular enzymes from the classes of hydrolases, such as proteases, (poly)esterases, lipases or lipolytic enzymes, amylases, cellulases or other glycosyl hydrolases, hemicellulase, cutinases, β-glucanases, oxidases, peroxidases, mannanases, perhydrolases, oxireductases and/or laccases. In order to ensure a particularly efficient removal of protein-containing and lipid-containing residues on the membrane, the use of at least one protease and/or lipase has proven to be particularly advantageous. The use of the enzymes in the method according to the invention surprisingly leads to an excellent cleaning effect without undesirable deposits caused by the enzymes occurring on the membrane.

Proteases of the subtilisin type and in particular proteases that can be obtained from Bacillus lentus are preferably used. Of particular interest are enzyme mixtures of, for example, protease and amylase, or of protease and lipase or lipolytic enzymes, or of protease and cellulase, or of cellulase and lipase or lipolytic enzymes, or of protease, amylase and lipase or lipolytic enzymes, or of protease, lipase or lipolytic enzymes and cellulase, in particular, however, protease and/or lipase-containing mixtures or mixtures with lipolytic enzymes. Examples of such lipolytic enzymes are the known cutinases. Suitable amylases include, in particular, α-amylases, iso-amylases, pullulanases and pectinases.

The amount of enzyme or enzymes in the cleaning liquid is preferably 0.01 to 10% by weight and more preferred 0.12 to 3% by weight, based on the total cleaning liquid. The enzymes are preferably used liquid enzyme formulation(s).

The cleaning liquid can furthermore contain at least one solvent, in particular alcohol. The cleaning liquid can contain precisely one solvent or a plurality of different solvents.

In order to further facilitate the removal of medicament residues from the membrane, it is preferred for the cleaning liquid to contain at least one water-soluble, organic solvent. In particular alcohols, such as ethanol, isopropanol, ethylene glycol, propylene glycol, glycerin or mixtures thereof are well suited as organic solvents herefor. In particular the use of ethanol is particularly preferred in view of its very good cleaning action, its bactericidal properties and its low toxicity. Since the organic solvents cited above are completely miscible with water, the content thereof in the cleaning liquid is not restricted and can be adapted to the respective contamination of the membrane.

The cleaning liquid preferably comprises not more than 50% by weight, more preferred not more than 30% by weight, and particularly preferred not more than 10% by weight of one or more solvents, in particular organic solvents, based on the total cleaning liquid.

The cleaning liquid can furthermore contain at least one disinfectant. The cleaning liquid can contain precisely one disinfectant or a plurality of different disinfectants.

In addition to quaternary ammonium salts and organic solvents, the cleaning solution can contain further disinfectants with an anti-microbial and anti-fungal effect. In particular chlorophene, biphenyl or chlorocresol are well suited for use in the cleaning solution. The use of at least one additional disinfectant is preferred in particular if the inhalation device is used by a plurality of patients in a medical environment.

The cleaning solution preferably contains at least one bleaching agent, with perborate-based, percarbonate-based or persulphate-based bleaching agents being particularly preferred. In particular sodium perborate, potassium peroxodisulphate and/or potassium hydrogen peroxomonosulphate as well as sodium percarbonate are well suited as such.

In order to achieve an additional improvement of the effect of the bleaching agent in the cleaning solution, in particular at temperatures below 40° C., the cleaning solution can furthermore contain one or more bleaching agent activators, such as tetra acetyl ethylene diamine (TAED).

Fragrances such as cinnamon oil, thyme oil, tea tree oil, almond oil, calendula, camomile extracts, peppermint extracts or eucalyptus extracts, menthol, amyl cinnamal, butylphenyl methylpropional, citronella oil, hexyl cinnamal or limonene, can furthermore be contained in the cleaning solution in order to give the inhalation therapy device a pleasant fragrance after the cleaning process.

The method according to the invention for cleaning an oscillatable membrane can comprise a plurality of successive steps or cycles of supplying a cleaning liquid to the membrane and of causing the membrane to oscillate such that the cleaning liquid is conveyed through the openings of the membrane. In particular two or more, three or more, or four or more such steps or cycles can be carried out.

According to one embodiment of the invention, in the method for cleaning the oscillatable membrane, the cleaning liquid is supplied to the membrane on the aerosol side and the membrane is caused to oscillate such that the cleaning liquid is conveyed through the openings of the membrane to the liquid side of the membrane.

The term “liquid side” hereby refers to that side of the membrane to which the liquid to be nebulised is supplied in the normal nebulising mode, i.e. when the inhalation therapy device is used for inhalation therapy, or on which the liquid to be nebulised is disposed in the normal nebulising mode. The term “aerosol side” refers to that side of the membrane on which the nebulised liquid, i.e. the aerosol generated owing to the oscillation of the membrane, exits.

In this case, the cleaning liquid flows through the membrane in the opposite direction as compared to aerosol generation in the normal nebulising mode. Owing to such a backwashing operation of the membrane, contaminations and blockages of the membrane can be removed in a particularly effective manner.

The cleaning liquid can furthermore contain at least one excipient, in particular a buffer, as was discussed in detail above. The cleaning liquid can contain precisely one excipient, in particular precisely one buffer, or a plurality of different excipients, in particular a plurality of different buffers.

The cleaning liquid can furthermore contain at least one water softener. The cleaning liquid can contain precisely one water softener or a plurality of different water softeners.

Owing to the use of a cleaning liquid additionally comprising at least one disinfectant and/or at least one water softener and/or at least one solvent, the efficiency of cleaning the oscillatable membrane can be further increased.

In one embodiment of the method according to the invention, the membrane is caused to oscillate in the same manner as the membrane is caused to oscillate in the nebulising mode, i.e. when using the inhalation therapy device for inhalation therapy. During cleaning of the membrane, the oscillations of the membrane can have the same frequency and/or amplitude as the oscillations of the membrane in the nebulising mode. This approach allows the cleaning method according to the invention to be performed in a particularly simple manner.

The cleaning method according to the invention can furthermore include a step of placing the membrane in an ultrasonic bath. This step can take place before or after the cleaning liquid is supplied to the membrane and the membrane is caused to oscillate.

The same cleaning liquid as is supplied to the membrane before it is caused to oscillate can be used for the ultrasonic bath, or a different cleaning liquid can be used.

According to a further aspect, the present invention provides a combination of a cleaning device and a cleaning liquid, the cleaning device being configured for cleaning an oscillatable membrane of a membrane aerosol generator of an inhalation therapy device. The cleaning device comprises a liquid supply device configured for supplying the cleaning liquid to the membrane, and an oscillation activating apparatus configured for activating membrane oscillations such that the cleaning liquid is conveyed through the openings of the membrane. The cleaning liquid contains at least one surfactant.

The combination can contain the cleaning liquid in liquid form or as a water-soluble substance, in particular a solid, such as in the form of one or more water-soluble tablets. In the latter case, the water-soluble substance is dissolved in water and the resulting solution is supplied to the membrane for cleaning.

The combination according to the invention of the cleaning device and the cleaning liquid is configured for carrying out the method according to the invention for cleaning an oscillatable membrane of a membrane aerosol generator of an inhalation therapy device. Consequently, the combination according to the invention offers the advantages already described above for the method according to the invention.

Furthermore, the features described above for the method according to the invention can also be used in the combination according to the invention. The cleaning liquid described above for use in the method according to the invention can in particular also be used for the combination according to the invention.

The oscillation activating apparatus can be configured such that it causes the membrane to oscillate in the same manner as the membrane is caused to oscillate in the nebulising mode.

The liquid supply device can be configured to supply the cleaning liquid to the aerosol side of the membrane. The oscillation activating apparatus can be configured to activate membrane oscillations such that the cleaning liquid is conveyed through the openings of the membrane to the liquid side of the membrane.

In one embodiment of the combination, the liquid supply device is a hollow body or in particular a hollow cylinder that is arranged with one end face on the membrane or the membrane aerosol generator such that a cleaning liquid filled into the hollow cylinder is disposed on the membrane.

The hollow cylinder can be configured as an elongated tube that is designed for insertion into a mouthpiece or mixing chamber of the inhalation therapy device.

The elongated tube can comprise a stop that limits insertion of the tube into the mouthpiece of an inhalation therapy device.

The elongated tube can extend in a tapered manner towards the end indented for arrangement on the membrane.

In one embodiment of the combination according to the invention, the liquid supply device is funnel-shaped and is arranged with an opening on the membrane or the membrane aerosol generator in such a manner that a cleaning liquid filled into the funnel-shaped liquid supply device is disposed on the membrane.

A seal can be provided on the liquid supply device, which prevents the escape of the cleaning liquid on the membrane or on the membrane aerosol generator.

A collecting receptacle can be provided for collecting the cleaning liquid exiting the membrane, in particular the cleaning liquid exiting on the liquid side of the membrane.

An accommodating member for the arrangement of the membrane aerosol generator can be provided on the collecting receptacle.

The liquid supply device and the collecting receptacle can be configured so as to be pivotable about an axis in relation to one another.

The liquid supply device can be configured integrally with or without a lid. The lid can be connected to the housing of the liquid supply device by means of a hinge, in particular a film hinge. The liquid supply device can consist of two halves, in which the membrane can be inserted, which are connected by a hinge, in particular a film hinge.

In a further embodiment, the liquid supply device can be configured as two pieces with or without a lid. The two halves of the liquid supply device, in which the membrane can be inserted, can be separated by a hinge.

The oscillation activating apparatus can be an actuating device that emits a preferably electric actuation signal for actuating the membrane aerosol generator.

The actuating device can be the control device of the inhalation therapy device.

The oscillation activating apparatus, such as the actuating device, in particular the control device of the inhalation therapy device, can be connected to the membrane aerosol generator by means of a cable. The oscillation activating apparatus can emit a preferably electric actuation signal to the membrane aerosol generator via the cable. The cable allows the membrane aerosol generator to be inserted into the cleaning device in an orientation or position that is rotated by 180° relative to the orientation or position in the normal nebulising mode. In this case, a backwashing mode is made possible in a simple manner, in which the cleaning liquid flows through the membrane in the opposite direction as compared to aerosol generation in the normal nebulising mode.

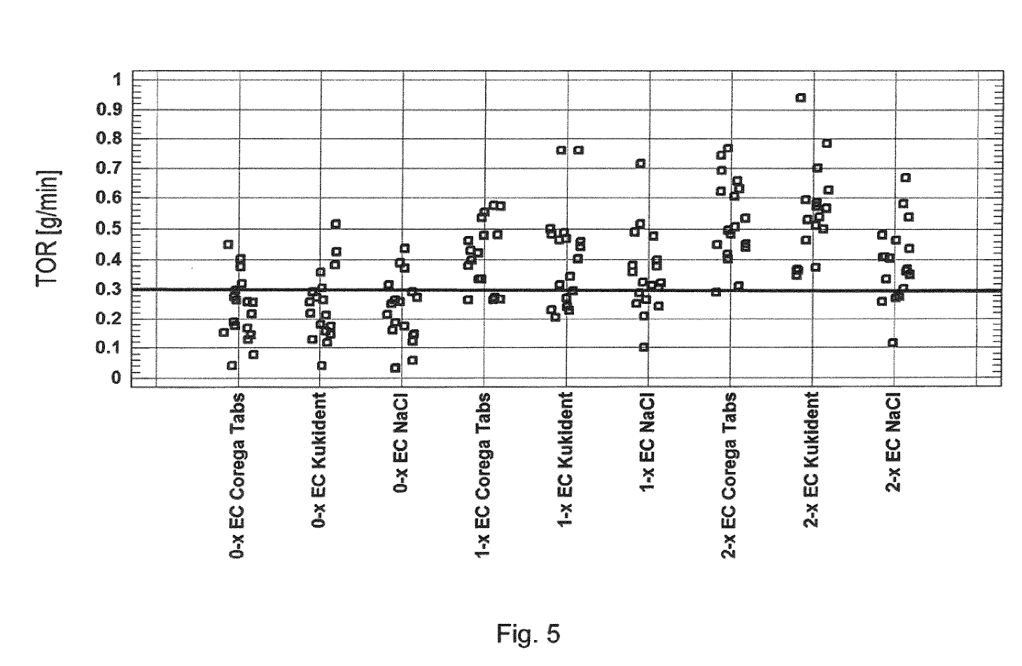

In one embodiment of the invention, the oscillation activating apparatus, such as the actuating device, in particular the control device of the inhalation therapy device, can be connected to the membrane aerosol generator in a wireless manner. The oscillation activating apparatus can be connected to the membrane aerosol generator in particular via a plug connection. In this case, the oscillation activating apparatus can emit a preferably electric actuation signal to the membrane aerosol generator via the plug connection, in particular via a plug contained in the plug connection. This plug connection is preferably configured to enable the membrane aerosol generator to be inserted into the cleaning device, in particular into the oscillation activating apparatus of the cleaning device, such as the control device of the inhalation therapy device, in an orientation or position that is rotated by 180° relative to the orientation or position in the normal nebulising mode, such that a backwashing operation can be performed in a simple manner. A cleaning device such as described in European patent application EP 14 156 519.2 can in particular be used, whereby the disclosure of EP 14 156 519.2 is incorporated herein in its entirety by reference thereto. The plug connection between the oscillation activating apparatus and the membrane aerosol generator can be configured in the manner described in EP 14 156 519.2 and shown in particular in FIGS. 5 to 8 of this patent application.

Such a connection of the oscillation activating apparatus, such as the actuating device, in particular the control device of the inhalation therapy device, with the membrane aerosol generator in a wireless manner, in particular by means of a plug connection, allows the cleaning method to be performed in a particularly simple and reliable manner. The actuation signal of the oscillation activating apparatus can correspond to the actuation signal that is supplied to the membrane aerosol generator in the nebulising mode, i.e. when using the inhalation therapy device for inhalation therapy.

The actuation signal of the oscillation activating apparatus can differ from the actuation signal of the nebulising mode in particular as regards amplitude and/or frequency and/or frequency change and/or duration and/or sequence of the activation periods.

The present invention will be described below, purely by way of example, by means of the enclosed figures, in which

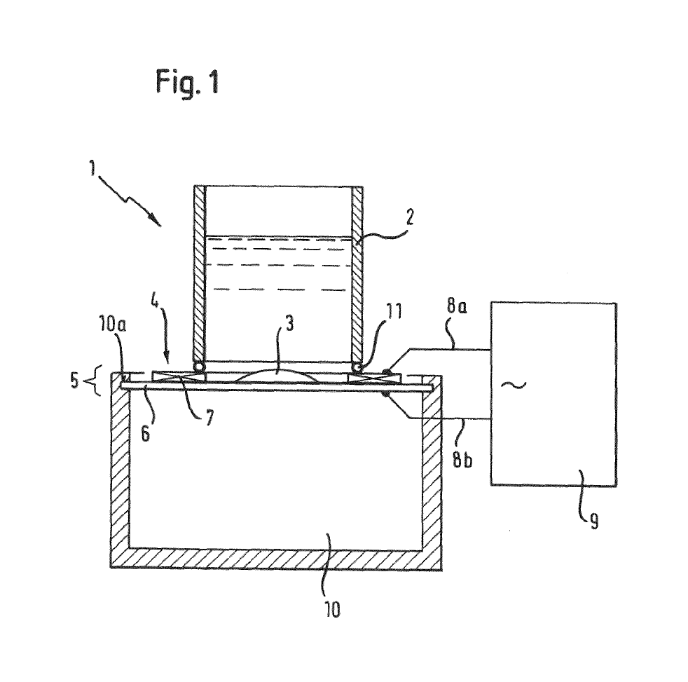

FIG. 1 shows a first embodiment of a cleaning device according to the invention, which is configured for cleaning an oscillatable membrane of an inhalation therapy device;

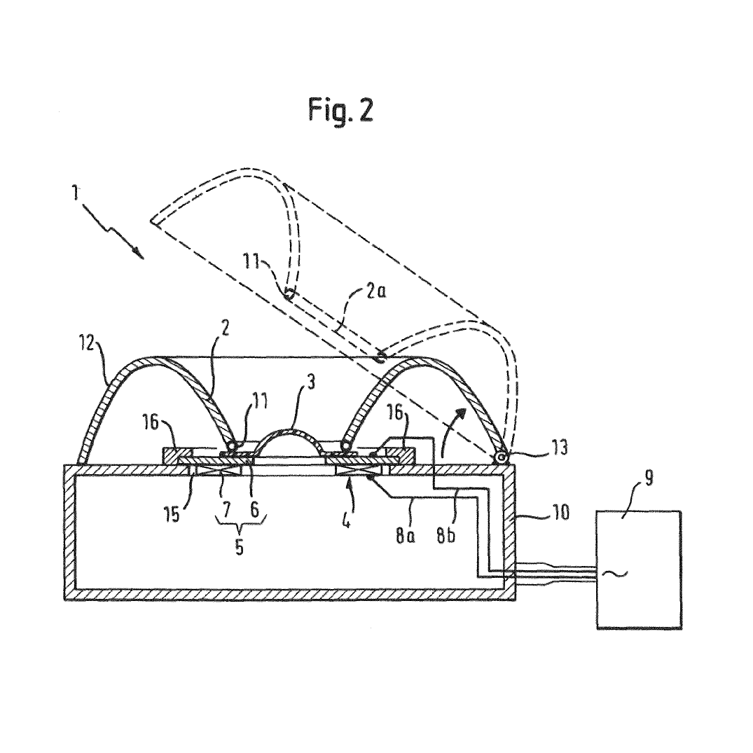

FIG. 2 shows a second embodiment of a cleaning device according to the invention;

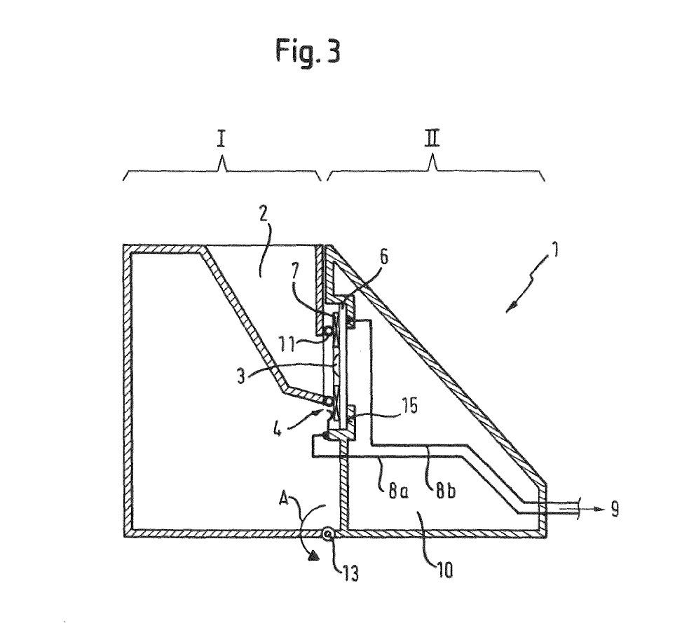

FIG. 3 shows a third embodiment of a cleaning device according to the invention;

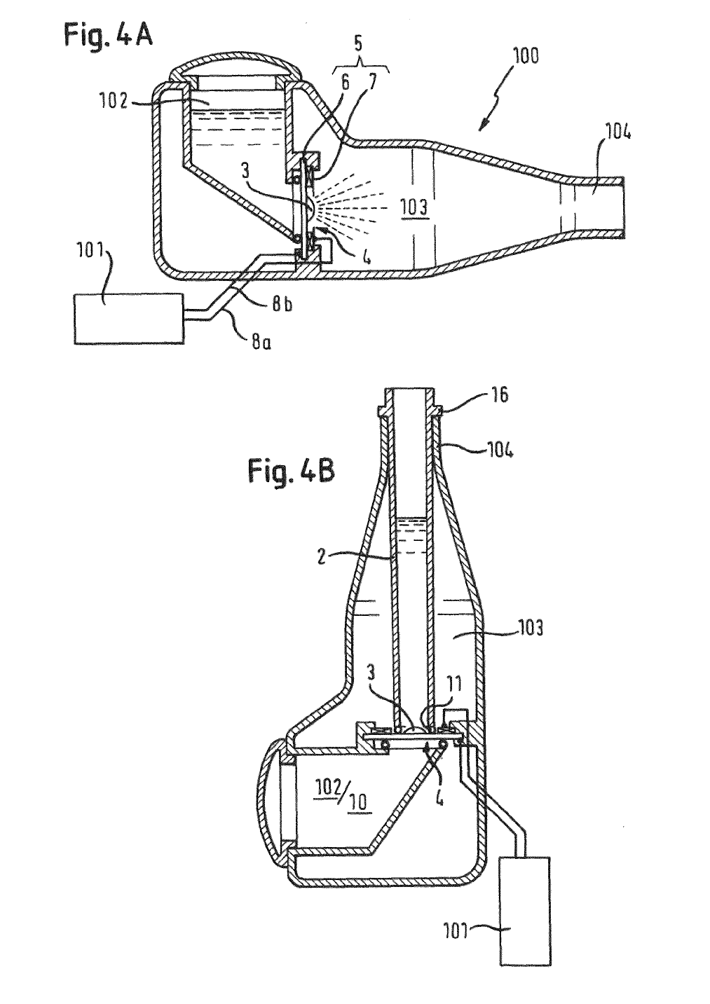

FIG. 4A shows an inhalation therapy device comprising a membrane aerosol generator;

FIG. 4B shows a fourth embodiment of a cleaning device according to the invention;

FIG. 5 shows experimental data for the nebulisation rates of membranes for different cleaning methods;

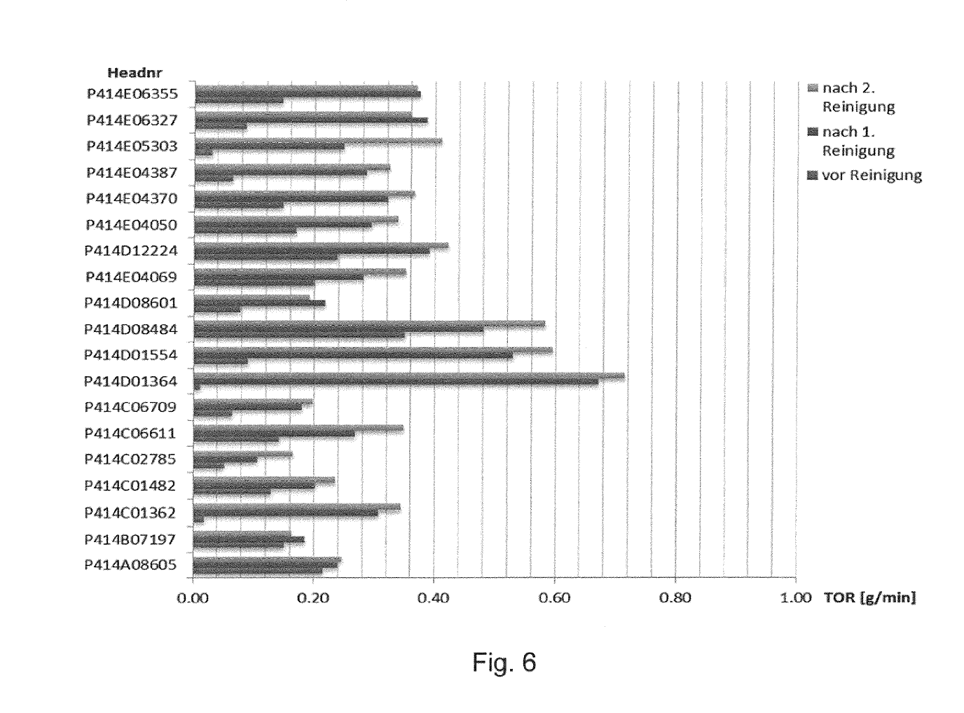

FIG. 6 shows experimental data for the nebulisation rates of membranes for cleaning with deionised water;

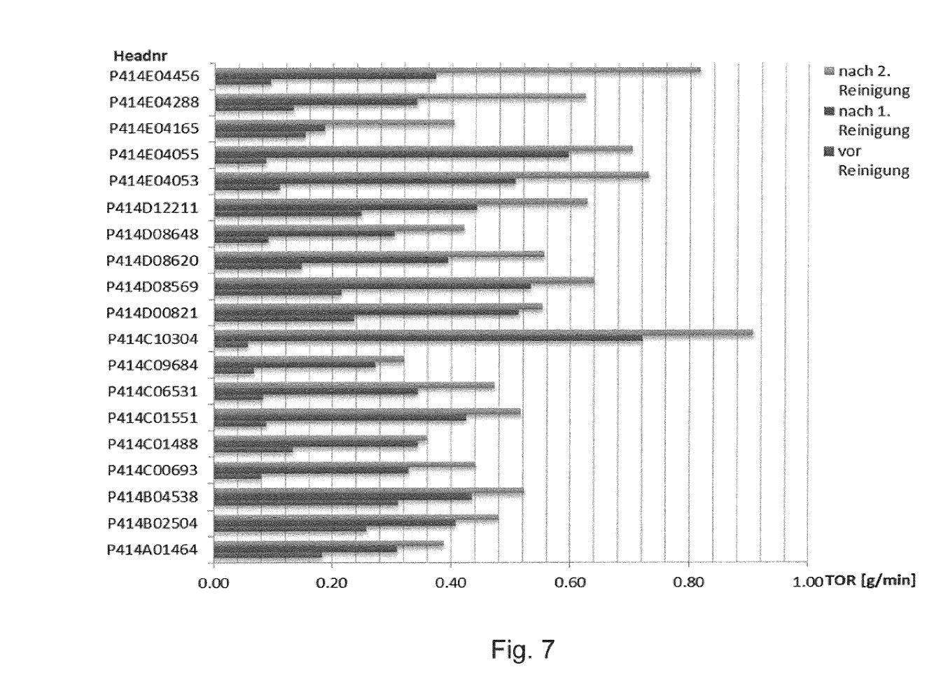

FIG. 7 shows experimental data for the nebulisation rates of membranes for cleaning with a Corega Tabs® solution;

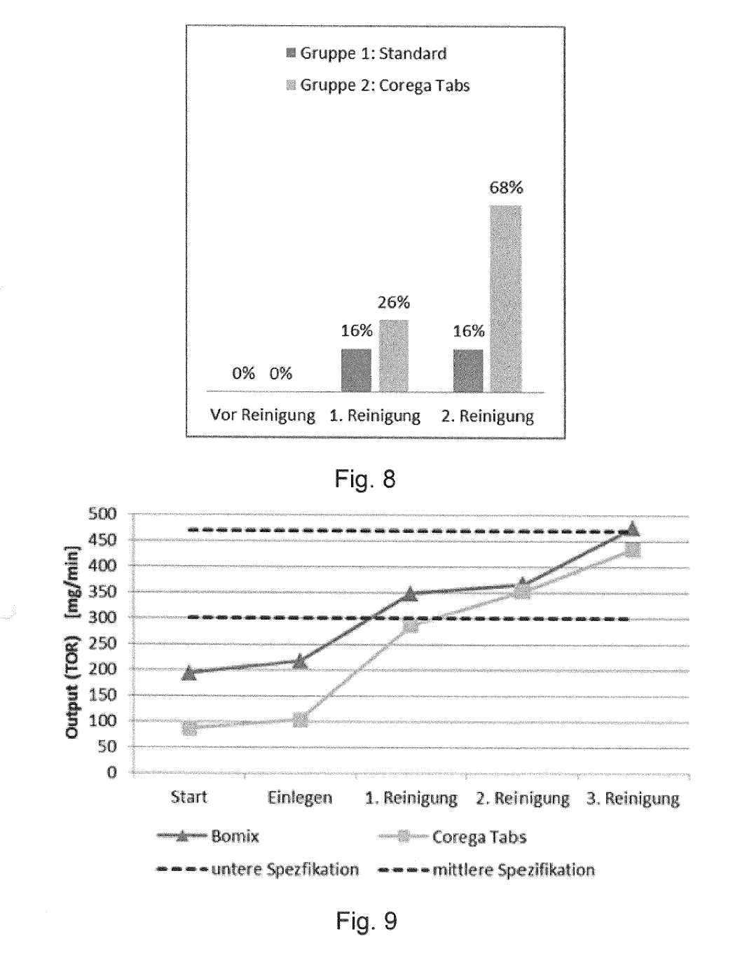

FIG. 8 shows a diagram in which the efficiency of cleaning with deionised water and the efficiency of cleaning with a Corega Tabs® solution are compared; and

FIG. 9 shows experimental data for the nebulisation rates of membranes for cleaning with Bomix® Plus and cleaning with a Corega Tabs® solution.

FIG. 1 shows a first advantageous embodiment of a cleaning device that is configured for cleaning an oscillatable membrane of a membrane aerosol generator of an inhalation therapy device and which can be used in the combination as according to the invention.

The shown cleaning device 1 comprises a liquid supply device 2 for supplying a cleaning liquid to a membrane 3. The cleaning liquid is supplied to that side of the membrane 3 on which the aerosol generated by the membrane aerosol generator is usually released during nebulisation of a liquid, i.e. to the aerosol side of the membrane.

In addition to the oscillatable membrane 3, the membrane aerosol generator 4 comprises an oscillation generator 5 that consists, in the shown embodiment, of an annular substrate 6 and an annular piezoelectric element 7 connected thereto. In the shown embodiment, the oscillation generator 5 thus has a rotationally symmetric basic structure, the axis of rotation of which lies in the drawing plane of FIG. 1.

As is furthermore shown by FIG. 1, connecting lines 8a, 8b are on the one hand connected with the oscillation generator 5 and are on the other hand connected with an oscillation activating apparatus 9 for activating oscillations of the membrane.

According to the embodiment shown in FIG. 1, the oscillation activating apparatus 9 supplies an electric actuation signal to the oscillation generator 5 of the membrane aerosol generator 4. When the actuation signal is supplied, the oscillatable membrane 3 is caused to oscillate by the oscillation generator 5 and the cleaning liquid is conveyed from the aerosol side of the membrane 3 through the membrane 3 to the liquid side of the membrane 3.

As is shown by FIG. 1, the cleaning liquid conveyed through the openings of the membrane is advantageously collected in a collecting receptacle 10, on which the membrane aerosol generator 4 is suitably arranged or attached. For example, according to FIG. 1, the annular substrate 6 is fixed at its outer periphery in a groove 10a of the collecting receptacle 10.

In the embodiment shown in FIG. 1, the liquid supply device 2 is realised in the form of a hollow cylinder that is open at both end faces. One of the end faces of the hollow cylinder 2 is positioned such that this end face is tightly sealed by the membrane 3 or the membrane aerosol generator 4. An annular seal 11 is advantageously arranged for this purpose on the end face that is facing the membrane, said seal abutting the membrane 3 or the membrane aerosol generator 4 in such a manner that a cleaning liquid filled into the hollow cylinder does not escape at the end face of the hollow cylinder that is facing the membrane but is rather disposed on the membrane 3. Provided that the hollow cylinder has a sufficient weight of its own to obtain the leak tightness (with or without seal 11) to be achieved for supply of the cleaning liquid, no further measures for fixing the liquid supply device 2 are necessary. However, fixings for the liquid supply device 2 can be alternatively (or additionally) provided, which reliably hold the liquid supply device 2 in the position suitable for supplying the cleaning liquid.

FIG. 2 shows a second advantageous embodiment of a cleaning device that can be used in the combination according to the invention. In the cleaning device 1 as shown in FIG. 2, the liquid supply device 2 for supplying a cleaning liquid to the aerosol side of the membrane 3 is formed in the shape of a funnel. Furthermore, the funnel-shaped liquid supply device 2 is configured integrally with a lid 12 that is fixed to the collecting receptacle 10 so as to be pivotable about an axis 13. In the closed state, the funnel-shaped liquid supply device 2 is positioned such that the free open end 2a abuts the membrane 3 or the membrane aerosol generator 4 so that a cleaning liquid filled into the funnel-shaped liquid supply device 2 is disposed on the membrane 3. A seal 11 is advantageously provided at the free open end of the liquid supply device 2 in order to prevent an undesirable escape of the cleaning liquid from the volume limited by the liquid supply device 2 and the membrane 3.

So that the membrane aerosol generator 4 can be removed from and inserted into the cleaning device 1 according to the second embodiment, the liquid supply device 2 can be pivoted together with the lid 12 about the axis 13. The membrane aerosol generator 4 is detachably fixed to an accommodating member 15 provided in the collecting receptacle, for example an adapted opening in the surface of the collecting receptacle 10 facing the lid, by means of one or more holding devices 16 that, for example, clamp the membrane aerosol generator 4.

As in the first embodiment, the cleaning liquid is also supplied in the second embodiment to the aerosol side of the membrane 3, i.e. to that side on which the aerosol generated by the membrane aerosol generator is usually released during nebulisation of a liquid. If the membrane aerosol generator 4 is activated and the membrane 3 is thus caused to oscillate, the cleaning liquid exits on the other side of the membrane 3, i.e. on that side on which the liquid to be nebulised is normally disposed in the nebulising mode (liquid side).

As in the first embodiment, an actuation signal is supplied to the membrane aerosol generator 4 by an oscillation activating apparatus 9 via connecting lines 8a and 8b.

FIG. 3 shows a third embodiment of a cleaning device 1 that can be used in the combination according to the invention. In this embodiment, the membrane aerosol generator 4 is arranged vertically rather than horizontally in the cleaning device 1 in order to demonstrate that the method according to the invention can also be carried out if the membrane 3 of the membrane aerosol generator 4 is not arranged horizontally. The membrane 3 of the membrane aerosol generator 4 can ultimately be arranged as desired provided that the cleaning liquid is supplied to the membrane 3 of the membrane aerosol generator 4, in particular to the aerosol side of the membrane 3, with the help of the liquid supply device 2, and the membrane 3 is caused to oscillate such that the cleaning liquid is conveyed through the openings of the membrane 3.

In the third embodiment shown in FIG. 3, the liquid supply device 2 is a container that is sloped in parts and has a vertical opening which is arranged such that the cleaning liquid is disposed on the aerosol side of the membrane 3.

A seal 11 is preferably provided at the opening of the liquid supply device 2, which prevents an undesirable escape of the cleaning liquid at the point of contact between the funnel/seal and the membrane/membrane aerosol generator.

As is indicated by the arrow A in FIG. 3, part I of the cleaning device 1, in which the liquid supply device 2 is formed, can be pivoted about a rotational axis, which exposes the accommodating member 15 for the membrane aerosol generator 4 that is formed in another part II of the cleaning device 1. The membrane aerosol generator 4 is placed in the accommodating member 15 and is thereby connected with connecting lines 8a and 8b, via which an actuation signal is supplied to the membrane aerosol generator 4. When the first housing part I of the cleaning device 1 is pivoted into the position shown in FIG. 3, the membrane aerosol generator 4 or the membrane 3 is fixed in the accommodating member since the seal 11 acts mechanically on the membrane aerosol generator 4. The second housing part II of the cleaning device 1 according to FIG. 3 serves as a collecting chamber 10 for the cleaning liquid conveyed through the membrane 3.

FIG. 4A shows an inhalation therapy device 100 with a membrane aerosol generator 4, which comprises, in addition to the membrane 3, an oscillation generator 5 having an annular substrate 6 and an annular piezoelectric element 7 that are connected with one another and with the membrane 3 in such a manner that the membrane 3 is caused to oscillate when the oscillation generator 5 is stimulated to oscillate by an electric actuation signal. The electric actuation signal is supplied via supply lines 8a, 8b that are connected to a control device 101 of the inhalation therapy device 100.

The liquid to be nebulised is filled into a liquid reservoir 102 of the inhalation therapy device 100 and is disposed on the liquid side of the membrane 3 in the nebulising mode. If an actuation signal is supplied to the membrane aerosol generator 4 by the control device 101, the membrane 3 is caused to oscillate, and the liquid to be nebulised is thus transported from the liquid side to the aerosol side of the membrane 3. The aerosol is released by the membrane aerosol generator 4 into a mixing chamber 103, from which a patient inhales the aerosol via a mouthpiece 104. Even though the mouthpiece 104 is shown integrally in FIG. 4A, it can, in many cases, be removed from the mixing chamber 103.

In an inhalation therapy device 100 configured in this manner, the cleaning device according to the invention comprises, as is shown in FIG. 4B, a liquid supply device 2 that is in the form of a tube and is inserted into the mouthpiece 104 of the inhalation therapy device 100. The length of the liquid supply device 2 depends on the size of the mouthpiece 104 and the mixing chamber 103 and is configured to be long enough that an open end face of the liquid supply device 2 reaches the membrane 3 such that the seal 11 provided on this end face abuts the membrane 3 or the membrane aerosol generator 4. If the mouthpiece 104 can be removed from the mixing chamber 103, the dimensions of the tube-like liquid supply device 2 can also be adapted to the mixing chamber 103.

The liquid supply device 2 preferably has a stop 16 that abuts the opening edge of the mouthpiece 104 or the mixing chamber 103 of the inhalation therapy device and thus prevents the liquid supply device 2 from being inserted too far into the inhalation therapy device 100, thereby damaging the membrane 3 or the membrane aerosol generator 4. Furthermore, the tube 2 is preferably tapered so as to facilitate insertion into the mouthpiece 104 or the mixing chamber 103 and to obtain at one end an end face cross-section that is adapted to the membrane 3 or the membrane aerosol generator 4 and to obtain at the opposite end an end face cross-section that is adapted to the opening of the mouthpiece 104 or the mixing chamber 103.

The cleaning liquid is filled into the tube 2 that forms the liquid supply device of the cleaning device according to the invention in the fourth embodiment in such a manner that said cleaning liquid is disposed on the aerosol side of the membrane 3 during the cleaning process. The membrane 3 is caused to oscillate by the control device 101, which is also used to actuate the membrane aerosol generator 4 for the generation of an aerosol. The control device 101 of the inhalation therapy device 100 is thus advantageously also used for cleaning. If an actuation signal is supplied to the membrane aerosol generator 4 by the control device 101, the membrane 3 is caused to oscillate, and thus the cleaning liquid supplied to the aerosol side of the membrane 3 by means of the liquid supply device 2 is transported through the membrane 3 and collected in the liquid reservoir 102. The liquid reservoir 102 of the inhalation therapy device 100 thereby serves as a collecting receptacle 10 for the cleaning liquid conveyed through the membrane 3.

However, in order to prevent the cleaning liquid collected in the liquid reservoir 102 from being inadvertently nebulised and inhaled, the lid of the liquid reservoir 102 should preferably be removed during cleaning. In order to collect the cleaning liquid then exiting the liquid reservoir 102, a separate collecting receptacle 10 must be provided. In this manner, the further advantage inherent to all of the embodiments according to the invention, i.e. that no cleaning liquid is inadvertently nebulised, is also reliably achieved in the embodiment shown in FIG. 4B.

It is of particular advantage in the last described fourth embodiment of a cleaning device according to the invention that the cleaning liquid is supplied to the aerosol side of the membrane 3 since it is prevented in this manner that a cleaning liquid filled into the liquid reservoir is inadvertently nebulised and inhaled. Supplying the cleaning liquid to the aerosol side of the membrane 3 prevents the inhalation therapy device 100 from possibly being used in the cleaning mode to nebulise a cleaning liquid.

Furthermore, the fourth embodiment is particularly advantageous since the control device 101 of the inhalation therapy device 100 is used as an oscillation activating apparatus 9 according to the invention in order to cause the membrane 3 to oscillate so that the cleaning liquid is transported from the aerosol side to the liquid side.

The control device of an inhalation therapy device can also be used in the previously described embodiments to supply the actuation signal to the membrane aerosol generator 4 so that the membrane 3 is caused to oscillate in order to bring about the desired transport of the cleaning liquid from the aerosol side to the liquid side of the membrane 3. However, in a cleaning device according to the invention, an actuating device that is independent of the inhalation therapy device can also be used as an oscillation activating apparatus, which provides the opportunity to supply other actuation signals, for example in other frequency ranges or with other waveforms. However, it has been shown that good cleaning results are achieved if the control device of the inhalation therapy device is used under the operating conditions that are also used for nebulisation of the liquid in order to generate an aerosol.

A highly effective method for cleaning an oscillatable membrane 3 of a membrane aerosol generator 4 of an inhalation therapy device 100 can be carried out with the combination according to the invention of a cleaning device and a cleaning liquid. For this purpose, a cleaning liquid is supplied, according to the invention, to the membrane 3, said cleaning liquid comprising at least one surfactant. Furthermore, the membrane 3 is caused to oscillate according to the invention in such a manner that the cleaning liquid is conveyed through the openings of the membrane 3.

Embodiments of the combination according to the invention comprise the embodiments of the cleaning device as described above together with a cleaning liquid that contains at least one surfactant. The cleaning liquid preferably further contains at least one enzyme and/or at least one acid. Such a combination of a cleaning device and a cleaning liquid can be used particularly advantageously to carry out the method as according to the invention.

A cleaning liquid comprising a surfactant, an enzyme and an acid can in particular be used for the combination according to the invention and the method according to the invention. In this case, contaminations and blockages of the membrane are removed in a particularly effective manner, as will be shown in detail below by means of experimental data.

FIG. 5 shows experimental data for the nebulisation rates (“Total Output Rate” [TOR]) of oscillatable membranes of membrane aerosol generators after using different cleaning methods. The data shown in FIG. 5 was collected for three groups comprising 18 membranes each, the nebulisation rates of which had decreased owing to contaminations and blockages of the membranes. The first three sets of data on the left-hand side of FIG. 5 (“0-x EC Corega Tabs®”; “0-x EC Kukident®”; “0-x EC NaCl”) show the nebulisation rates of the groups of membranes in the untreated state, i.e. before cleaning is carried out. As is apparent from FIG. 5, the nebulisation rates of these membrane groups are substantially in the same range.

The nebulisation rates (TOR) were gravimetrically determined using isotonic saline solution (NaCl 0.9%). This weighing could alternatively be measured using medicaments or test liquids.

Each of the three aforementioned groups of membranes was cleaned using a different cleaning method, i.e. using a different cleaning liquid. A cleaning liquid was in particular supplied to the aerosol side of all the membranes, and the membranes were caused to oscillate such that the cleaning liquid was conveyed though the openings of the membranes to the liquid side of the membranes. A Corega Tabs® solution was used as the cleaning liquid for the first group of membranes, a Kukident® solution was used for the second group of membranes and a 0.9% NaCl solution was used for the third group of membranes. For the Corega Tabs® solution, one tablet of the denture cleaning agent Corega Tabs® Bio Formula by GlaxoSmithKline was dissolved in tap water. For the Kukident® solution, one tablet of the denture cleaning agent Kukident by Reckitt Benckiser was dissolved in tap water.

The nebulisation rates of the three groups of membranes following one such cleaning cycle are shown in the middle of FIG. 5 (“1-x EC Corega Tabs®”; “1-x EC Kukident®”; “1-x EC NaCl”). As is apparent from FIG. 5, the membranes cleaned with the Corega Tabs® solution and the membranes cleaned with the Kukident® solution have a higher nebulisation rate than the membranes cleaned with the NaCl solution.

Finally, a second cleaning cycle was carried out in the manner as described above. The nebulisation rates achieved after this second cleaning cycle are shown on the right-hand side of FIG. 5 (“2-x EC Corega Tabs®”; “2-x EC Kukident®”; “2-x EC NaCl”). As is apparent from FIG. 5, a further improvement of the nebulisation rates was achieved as a result of the second cleaning cycle for the membranes cleaned with the Corega Tabs® solution and the Kukident® solution as compared to the membranes cleaned with the NaCl solution.

The increase in cleaning efficiency achieved by using the Corega Tabs® solution or the Kukident® solution as compared to cleaning with the NaCl solution is also apparent from the following tables 1 to 3.

Table 1 shows the proportion of membranes from each of the three aforementioned membrane groups having a nebulisation rate of ≥300 mg/min (lower specification; solid horizontal line in FIG. 5 at TOR=0.3 g/min). Table 2 shows the proportion of membranes from each of the three aforementioned membrane groups having a nebulisation rate of ≥470 mg/min (middle specification). Table 3 shows the mean values and deviations of the experimental data shown in FIG. 5.

TABLE 1
Proportion of Membranes with TOR ≥ 300 mg/min

| | NaCl solution | Corega Tabs | Kukident |
|---|---|---|---|
| Initial values | 22% | 22% | 28% |
| 1^{st} cleaning | 67% | 78% | 67% |
| 2^{nd} cleaning | 78% | 94% | 100% |

TABLE 2
Proportion of Membranes with TOR ≥ 470 mg/min

| | NaCl solution | Corega Tabs | Kukident |
|---|---|---|---|
| Initial values | 0% | 0% | 6% |
| 1^{st} cleaning | 22% | 33% | 28% |
| 2^{nd} cleaning | 22% | 64% | 67% |

TABLE 3
Mean values (deviations) TOR in g/min

| | NaCl solution | Corega Tabs | Kukident |
|---|---|---|---|
| Initial values | 0.226 | 0.232 | 0.246 |
| | (+/−0.110) | (+/−0.110) | (+/−0.119) |
| 1^{st} cleaning | 0.350 | 0.410 | 0.406 |
| | (+/−0.137) | (+/−0.107) | (+/−0.163) |
| 2^{nd} cleaning | 0.388 | 0.525 | 0.538 |
| | (+/−0.129) | (+/−0.138) | (+/−0.157) |

It is in particular apparent from Table 1 that when cleaning with the Kukident® solution all of the membranes, and when cleaning with the Corega Tabs® solution almost all of the membranes had a nebulisation rate of ≥300 mg/min after the second cleaning cycle. Furthermore, the proportion of membranes having a nebulisation rate of ≥470 mg/min when cleaned with the Corega Tabs® solution or the Kukident® solution is approximately three times as high following the second cleaning cycle as when cleaning was carried out with the NaCl solution (see Table 2). The increase in efficiency of the cleaning method by using the Corega Tabs® solution or the Kukident® solution as compared to cleaning with the NaCl solution is also apparent from Table 3.

In the present invention, the following TOR specifications can generally exist for the membrane.

For particle sizes (MMAD) of 2.0 μm to 4.0 μm, the specification can be 100 mg/min, preferably 200 mg/min and more preferred 300 mg/min.

For an MMAD of 3.0 μm to 5.0 μm, the specification can be 300 mg/min, preferably 400 mg/min and more preferred 500 mg/min.

For an MMAD of 3.5 μm to 6.5 μm, the specification can be 600 mg/min, preferably 700 mg/min and more preferred 800 mg/min.

In a further experimental examination, two groups having 19 membranes each, the nebulisation rates of which had decreased owing to contaminations and blockages, were subjected to cleaning with deionised water (first group) or cleaning with a Corega Tabs® solution (second group). The experimental results for the first and second groups of membranes are shown in FIG. 6 and FIG. 7. In these figures, the vertical axis shows the identification numbers (head numbers) of the membranes whilst the horizontal axis shows the nebulisation rates (“TOR”) in g/min.

In FIGS. 6 and 7, the lower bar for each membrane identification number shows the nebulisation rate prior to cleaning, the middle bar shows the nebulisation rate after the first cleaning cycle and the upper bar shows the nebulisation rate after the second cleaning cycle. The first and second cleaning cycles were carried out in the manner already described above in connection with FIG. 5.

As is apparent from a comparison of FIG. 6 with FIG. 7, the efficiency of the cleaning method is significantly increased by using the Corega Tabs® solution as compared to cleaning with deionised water. This is also apparent from FIG. 8, which shows the percentage of membranes of the first and second group having a nebulisation rate of ≥470 mg/min.

As is apparent from FIG. 8, a significantly more efficient cleaning of the membranes could be achieved when using the Corega Tabs® solution than could be achieved when using deionised water. In particular, after the second cleaning cycle, 68% of the membranes cleaned with the Corega Tabs® solution had a nebulisation rate of ≥470 mg/min, i.e. a nebulisation rate equal to or greater than the middle specification.

FIG. 9 shows experimental data for a first membrane (upper curve in FIG. 9) and a second membrane (lower curve in FIG. 9), the nebulisation rates of which had decreased owing to contaminations and blockages. These two membranes were cleaned in substantially the same manner as already discussed above in connection with FIG. 5, whereby the membranes were placed into the cleaning liquid prior to the first cleaning cycle and a third cleaning cycle was carried out after the second cleaning cycle. Bomix® Plus by Paul Hartmann AG, which contains a disinfectant, was used as the cleaning liquid for the first membrane. A Corega Tabs® solution as described above was used as the cleaning liquid for the second membrane.

As is apparent from FIG. 9, a significant increase in the nebulisation rate to a value of approximately 450 mg/min could be achieved after the third cleaning cycle owing to cleaning with Bomix® Plus and cleaning with the Corega Tabs® solution. Furthermore, the nebulisation rates of the membranes were already greater than (first membrane) or close to (second membrane) the lower specification of 300 mg/min even after the first cleaning cycle.

The invention is not limited to the described embodiments but can be modified within the scope of the following patent claims.

1. Method for cleaning an oscillatable membrane of a membrane aerosol generator of an inhalation therapy device, wherein
a cleaning liquid is supplied to the membrane, and
the membrane is caused to oscillate such that the cleaning liquid is conveyed through the openings of the membrane, whereby
the cleaning liquid contains at least one surfactant.

2. Method according to claim 1, wherein the cleaning liquid further contains at least one enzyme and/or at least one acid.

3. Method according to claim 2, wherein the at least one acid is an organic acid, in particular citric acid.

4. Method according to claim 2, wherein the at least one enzyme is an enzyme for cleaving proteins and/or peptides.

5. Method according to claim 1, wherein
the cleaning liquid is supplied to the membrane on the aerosol side, and
the membrane is caused to oscillate such that the cleaning liquid is conveyed through the openings of the membrane to the liquid side of the membrane.

6. Method according to claim 1, wherein the cleaning liquid further contains at least one disinfectant and/or at least one water softening agent and/or at least one solvent.

7. Method according to claim 1, wherein the cleaning liquid further contains at least one excipient, in particular a buffer.

8. Method according to claim 1, wherein the membrane is caused to oscillate in the same manner as the membrane is caused to oscillate in the nebulising mode.

9. Combination of a cleaning device and a cleaning liquid, wherein the cleaning device is configured for cleaning an oscillatable membrane of a membrane aerosol generator of an inhalation therapy device and comprises:
a liquid supply device configured for supplying the cleaning liquid to the membrane, and
an oscillation activating apparatus configured for activating membrane oscillations such that the cleaning liquid is conveyed through the openings of the membrane, whereby
the cleaning liquid contains at least one surfactant.

10. Combination according to claim 9, wherein the cleaning liquid further contains at least one enzyme and/or at least one acid.

11. Combination according to claim 9, wherein
the liquid supply device is configured for supplying the cleaning liquid to the aerosol side of the membrane, and
the oscillation activating apparatus is configured for activating membrane oscillations such that the cleaning liquid is conveyed through the openings of the membrane to the liquid side of the membrane.

12. Combination according to claim 9, which is configured for carrying out the method according to claim 1.

13. Combination according to claim 9, wherein the liquid supply device is a hollow cylinder that is arranged with one end face on the membrane or the membrane aerosol generator such that a cleaning liquid filled into the hollow cylinder is disposed on the membrane.

14. Combination according to claim 13, wherein the hollow cylinder is configured as an elongated tube that is designed for insertion into a mouthpiece or a mixing chamber of the inhalation therapy device.

15. Combination according to claim 9, wherein the liquid supply device is funnel-shaped and is arranged with an opening on the membrane or the membrane aerosol generator in such a manner that a cleaning liquid filled into the funnel-shaped liquid supply device is disposed on the membrane.

16. Method according to claim 3, wherein the at least one enzyme is an enzyme for cleaving proteins and/or peptides.

17. Combination according to claim 10, wherein
the liquid supply device is configured for supplying the cleaning liquid to the aerosol side of the membrane, and
the oscillation activating apparatus is configured for activating membrane oscillations such that the cleaning liquid is conveyed through the openings of the membrane to the liquid side of the membrane.

18. Combination according to claim 10, wherein the liquid supply device is a hollow cylinder that is arranged with one end face on the membrane or the membrane aerosol generator such that a cleaning liquid filled into the hollow cylinder is disposed on the membrane.

19. Combination according to claim 10, wherein the hollow cylinder is configured as an elongated tube that is designed for insertion into a mouthpiece or a mixing chamber of the inhalation therapy device.

20. Combination according to claim 10, wherein the liquid supply device is funnel-shaped and is arranged with an opening on the membrane or the membrane aerosol generator in such a manner that a cleaning liquid filled into the funnel-shaped liquid supply device is disposed on the membrane.
